(19) United States
(12) Patent Application Publication
(10) Pub. No.: US 2017/0275219 A1
(43) Pub. Date: Sep. 28, 2017

(54) SYSTEMS AND METHODS FOR DEHYDROGENATION OF ALKANES

(71) Applicant: SABIC Global Technologies B.V., Bergen op Zoom (NL)

(72) Inventors: Zeeshan Nawaz, Riyadh (SA); Faisal Baksh, Manama (BH); Adel Abdullah Al-Ghamdi, Riyadh (SA)

(73) Assignee: SAVIC Global Technologies B.V., Bergen op Zoom (NL)

(21) Appl. No.: 15/504,979

(22) PCT Filed: Aug. 17, 2015
(86) PCT No.: PCT/IB2015/056256

§ 371 Date: Feb. 17, 2017

Related U.S. Application Data
(60) Provisional application No. 62/040,249, filed on Aug. 21, 2014.

(51) Int. Cl. C07C 5/333

(52) U.S. Cl. CPC C07C 5/3337; C07C 5/3335; C07C 2523/26; C07C 2523/42; C07C 2523/652

(57) ABSTRACT

The presently disclosed subject matter relates to methods and systems for alkane dehydrogenation. In a particular non-limiting embodiment, the presently disclosed subject matter provides a system for the dehydrogenation of alkanes that includes two or more reactors configured to perform a dehydrogenation reaction of an alkane in the presence of a catalyst to produce an olefin and a catalyst regenerator, coupled to each of the two or more reactors through at least one transfer line to a regenerator, for the regeneration of spent catalyst.

### FIELD

The presently disclosed subject matter relates to methods and systems for alkane dehydrogenation using two or more reactors.

### BACKGROUND

Alkane dehydrogenation is a process in which saturated hydrocarbons are converted to unsaturated hydrocarbons (e.g., olefins) and hydrogen (H₂). Olefins produced by dehydrogenation of alkanes can be useful as intermediates in the production of other hydrocarbon conversion products, such as propylene glycol and fuel. Examples of alkane dehydrogenation processes can include conversion of ethane to ethylene, propane to propylene, iso-butane to iso-butylene, ethylbenzene to styrene and C₆ to C₁₉ alkanes to the corresponding C₆ to C₁₉ monoolefins.

Dehydrogenation of alkanes to olefins can generally be classified as either oxidative or non-oxidative reactions. Disadvantages associated with oxidative dehydrogenation include high exothermicity and low desired product selectivity and quality. Non-oxidative processes (i.e., direct dehydrogenation or catalylic dehydrogenation) can suffer from the requirement of a continuous heat supply to initiate the endothermic reaction. The temperatures that are required to shift the equilibria favorably to alkene products during direct dehydrogenation can promote rapid deactivation of the catalyst by coking, resulting in the need for frequent catalyst regeneration. These high temperatures can also lead to thermal cracking of the alkanes, which can lead to undesirable non-selective side reactions that result in formation of byproducts.

Certain systems for performing alkane dehydrogenation reactions are known in the art. Chinese Patent Application No. CN1962573 discloses systems for the production of propylene by steam cracking, where the system includes a regenerator and a plurality of fluidized bed reactors arranged in series to perform distinct stages of a steam cracking reaction. Chinese Patent No. CN101168681 discloses systems for producing olefins that include a regenerator and a fluidized bed reactor that contains multiple catalyst beds. U.S. Pat. No. 7,347,930 discloses a system that includes a reactor with multiple reaction zones or multiple reactors, in which each reaction zone or reactor is configured to perform different stages of a reaction. U.S. Pat. No. 7,029,571 discloses methods for the production of C₈ to C₂₈ hydrocarbons using systems that include multiple fluidized bed reactors.

Therefore, there remains a need in the art for techniques for performing alkane dehydrogenation reactions that increase productivity by enhancing heat stability of the system, enhancing catalyst circulation and minimizing the hydrodynamic issues that can occur during alkane dehydrogenation.

### SUMMARY OF THE DISCLOSED SUBJECT MATTER

Disclosed, in various embodiments, are systems for the catalytic dehydrogenation of alkanes and methods for producing an olefin.

A system for the catalytic dehydrogenation of alkanes, comprises: two or more reactors configured to perform a dehydrogenation reaction of an alkane in the presence of a catalyst to produce an olefin; and a catalyst regenerator, coupled to each of the two or more reactors through at least one spent catalyst transfer line, for regeneration of spent catalyst transferred from the two or more reactors.

A method for producing an olefin, comprises: feeding a hydrocarbon feedstream comprising an alkane into two or more reactors; reacting the hydrocarbon feedstream with a catalyst to produce an olefin in each of the two or more reactors through a dehydrogenation reaction; removing and transferring spent catalyst from the two or more reactors to a catalyst regenerator; regenerating the spent catalyst in the catalyst regenerator to obtain a regenerated catalyst; and transferring the regenerated catalyst to the two or more reactors to be used in the dehydrogenation reaction.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram depicting an exemplary system for the dehydrogenation of an alkane in accordance with one non-limiting embodiment of the disclosed subject matter.

FIG. 2 is a schematic diagram depicting an exemplary catalyst regenerator for regeneration of spent catalyst during the alkane dehydrogenation process in accordance with one non-limiting embodiment of the disclosed subject matter.

FIG. 3 is a schematic diagram depicting an exemplary method for the dehydrogenation of an alkane in accordance with one non-limiting embodiment of the disclosed subject matter.

### DETAILED DESCRIPTION

The present disclosure provides systems and methods for the dehydrogenation of alkanes to unsaturated hydrocarbons. For example, the presently disclosed subject matter provides for systems and methods for the dehydrogenation of alkanes, including an integrated system that includes two or more reactors and a catalyst regenerator.

In certain embodiments, systems of the disclosed subject matter can include two or more reactors configured to perform a dehydrogenation reaction of an alkane in the presence of a catalyst to produce an olefin. In certain embodiments, the two or more reactors can be fluidized bed reactors. In certain embodiments, the two or more reactors can be configured to produce different olefins.

The system can further include a catalyst regenerator coupled to the two or more reactors. In certain embodiments, the catalyst regenerator can be coupled to each of the two or more fluidized bed reactors through at least one spent catalyst transfer line. The spent catalyst transfer line can transport the spent, e.g., inactivated and/or coked, catalyst from the two or more reactors to the catalyst regenerator. In certain embodiments, the system can further include two or more transfer lines for transporting the regenerated catalyst from the catalyst regenerator to the two or more reactors to continue the dehydrogenation reaction.

In certain embodiments, methods of the disclosed subject matter can include feeding a hydrocarbon feedstream into the two or more reactors and reacting the hydrocarbon feedstream with a catalyst to produce an olefin through a dehydrogenation reaction. In certain embodiments, the feedstream can include an alkane, a lower alkane, propane, butane, iso-butane, or a combination comprising at least one of the foregoing. In certain embodiments, the olefin product can include an alkene, a lower alkene, propylene, butylene (butene), iso-butene or a combination comprising at least one of the foregoing. In certain embodiments, the hydrocarbon feedstream to be fed into the reactors includes iso-butane.

In certain embodiments, dehydrogenation of iso-butane into iso-butylene, in the presence of a catalyst, is performed in the two or more reactors. In certain embodiments, dehydrogenation of propane into propylene, in the presence of a catalyst, is performed in the two or more reactors.

In certain embodiments, the method can further include removing and transferring spent catalyst from the two or more reactors to a catalyst regenerator. The method can further include contacting the spent catalyst with a regeneration gas in the catalyst regenerator to obtain a regenerated catalyst. In certain embodiments, the method can include transferring the regenerated catalyst to the two or more reactors to catalyze the alkane dehydrogenation reaction.

In certain embodiments, the catalyst that can be used in the systems and/or methods of the present disclosure can include a metal and/or metal oxide. Non-limiting examples of metals include platinum, chromium or a combination comprising at least one of the foregoing. The catalyst can further include a support such as alumina, silica, titania or a combination comprising at least one of the foregoing.

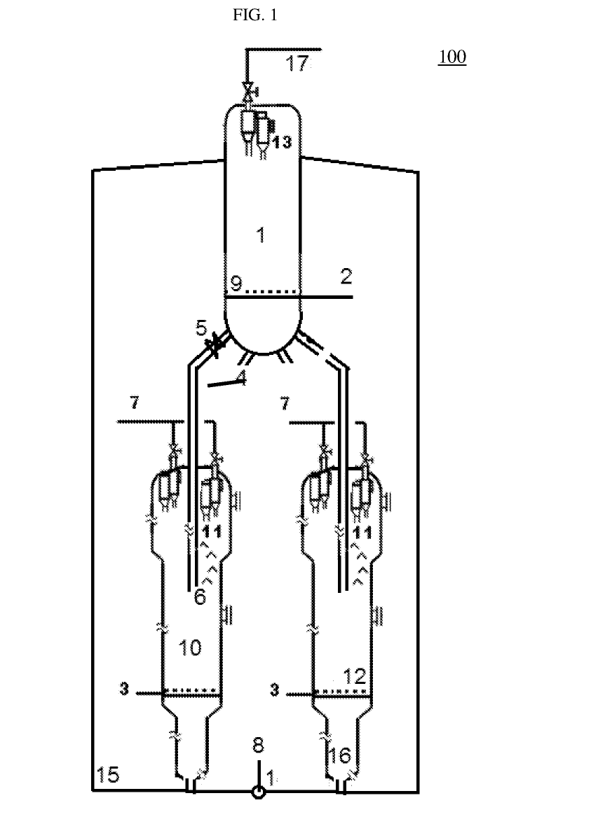

For the purpose of illustration and not limitation, FIG. 1 is a schematic representation of an exemplary system according to the disclosed subject matter. In certain embodiments, the system 100 can include two or more reactors 10 configured to perform an alkane dehydrogenation reaction to produce an olefin product. For example, but not by way of limitation, the system 100 can include two, three, four, five, six, seven, eight or more reactors. The presently disclosed subject matter does not include systems that include a single reactor and a single regenerator. The presently disclosed subject matter is directed to a system that includes at least two reactors coupled to a single catalyst regenerator.

In certain embodiments, the two or more reactors can be riser reactors, fixed bed reactors, such as multi-tubular fixed bed reactors, fluidized bed reactors, such as entrained fluidized bed reactors and fixed fluidized bed reactors, and slurry bed reactors such as three- phase slurry bubble columns and ebullated bed reactors, or a combination comprising at least one of the foregoing. In certain embodiments, the two or more reactors of the system can be different types of reactors and/or configured to produce different olefin products.

In certain embodiments, the two or more reactors can be fluidized bed reactors. A fluidized bed reactor includes one or catalyst beds containing catalyst particles that are fluidized by the feedstream, i.e., reactants of the alkane dehydrogenation reaction. In certain embodiments, each of the fluidized bed reactors of the presently disclosed subject matter can include one catalyst bed.

In certain embodiments, the system 100 can include one or more one feed lines 3 to introduce a hydrocarbon feedstream into each of the two or more reactors 10. The one or more feed lines 3 can be disposed at any part of the reactor 10. For example, a feed line 3 can be disposed on the side or proximate to the bottom of the reactors 10. In certain embodiments, a feed line 3 can be connected to a distributor 12, which can distribute the hydrocarbon feedstream throughout the reactor.

In certain embodiments, each of the reactors can further include one or more cyclones 11. The one or more cyclones 11 can be used to separate the chemical product from the catalyst and to further remove the chemical product from the reactor 10 through a product discharge line 7. In certain embodiments, the product discharge line 7 can be coupled to another reactor that uses the olefin product as a reactant. For example, and not by way of limitation, the product discharge line 7 can be coupled to a methyl tertiary butyl ether (MTBE) reactor, which can use the olefin iso-butylene as a reactant.

The system 100 can further include one or more transfer lines 15, e.g., spent (coked) catalyst transfer lines, connecting the one or more catalyst regenerators 1 of the system to the two or more reactors 10 of the system 100. The transfer lines 15 can function to transport the spent catalyst to the catalyst regenerator 1 from the two or more reactors 10. In certain embodiments, the transfer lines 15 can be disposed at any part of the two or more reactors 10. For example, and not by way of limitation, the transfer lines 15 can be positioned external to the individual reactors. In certain embodiments, a transfer line 15 can be positioned at the bottom of the reactor 10. In certain embodiments, the transfer lines 15 can be disposed at any part of the catalyst regenerator 1. In certain embodiments, the transfer lines 15 can be positioned at the sides and/or bottom of the catalyst regenerator 1. In certain embodiments, the transfer lines 15 can collect the spent catalyst from a reactor and transfer the spent catalyst to the regenerator 1. In certain embodiments, the system 100 can include one or more transfer lines 4, e.g., regenerated catalyst transfer lines, to transfer the regenerated catalyst from the catalyst regenerator 1 to the reactors 10.

In certain embodiments, the one or more transfer lines 15 can be further coupled to an inlet gas line 8 that feeds a lift or carrier gas into the transfer line 15 to transport the spent catalyst from the reactors 10 to the catalyst regenerator 1. The lift or carrier gas can include, but is not limited to, natural gas, air, oxygen-rich gas, oxygen-lean gas, carbon monoxide, carbon dioxide, nitrogen, steam combustion or exhaust gas, or a combination comprising at least one of the foregoing. In certain embodiments, the lift or carrier gas can include air. In certain embodiments, where lift gas is used within the one or more regenerated catalyst transfer lines 4 to transfer the regenerated catalyst from the catalyst regenerator 1 to the reactors 10, the lift gas can include hydrogen, natural gas, unsaturated hydrocarbons, saturated hydrocarbons or a combination comprising at least one of the foregoing. For example, and not by way of limitation, the lift gas for transporting regenerated catalyst to the reactors can include one or more hydrocarbons that are included in the feedstream and/or to be used in the reactor.

“Coupled” as used herein refers to the connection of a system component to another system component by any means known in the art. The type of coupling used to connect two or more system components can depend on the scale and operability of the system. For example, and not by way of limitation, coupling of two or more components of a system can include one or more joints, valves, fitting, coupling or sealing elements. Non-limiting examples of joints include threaded joints, soldered joints, welded joints, compression joints and mechanical joints. Non-limiting examples of fittings include coupling fittings, reducing coupling fittings, union fittings, tee fittings, cross fittings and flange fittings. Non-limiting examples of valves include gate valves, globe valves, ball valves, butterfly valves and check valves.

In certain embodiments, the system 100 can include one or more catalyst strippers 16 to remove adsorbed hydrocarbons, e.g., coke, from the surface of spent catalyst prior to the transfer of the spent catalyst to the catalyst regenerator. In certain embodiments, the stripping of the adsorbed hydrocarbons can include contacting the spent catalyst with a stripping gas such as hydrogen, nitrogen or a combination comprising at least one of the foregoing, within the stripper. In certain embodiments, a reactor stripper 16 of the presently disclosed subject matter can be coupled to a reactor 10, e.g., coupled to the bottom of a reactor. The reactor stripper 16 coupled to each of the two or more reactors 10 of the presently disclosed subject matter can be any stripper known to one of ordinary skill in the art. For example, but not by way of limitation, U.S. Pat. Nos. 6,248,298 and 7,744,746, and U.S. Patent Application No. 2011/0114468, incorporated herein by reference in their entireties, disclose strippers that can be used in the presently disclosed subject matter. The dimensions and structure of the reactor stripper 16 of the presently disclosed subject matter can vary depending on the physical size of the reactor 10 and the capacity of the reactor 10. In certain embodiments, the one or more transfer lines 15 can transfer the spent catalyst from a stripper 16 that is coupled to the reactor 10 to the catalyst regenerator 1.

The dimensions scale and structure of each of the reactors 10 contained within the system 100 of the present disclosure can depend on the desired hydrocarbon feedstream. In addition, the dimensions and structure of a reactor can vary depending on the capacity of the reactor. The capacity of the reactor can be determined by the reaction rate, which can depend on the active metal content of the catalyst, and the stoichiometric quantities of the reactants. In certain embodiments, a reactor of the presently disclosed subject matter can have a capacity of about 1 ton per hour to about 200 tons per hour. For example, and not by way of limitation, the reactor can have a capacity of about 1 ton/hour to about 5 tons/hour, of about 1 ton/hour to about 10 tons/hour, of about 1 ton/hour to about 20 tons/hour, of about 1 ton/hour to about 30 tons/hour, of about 1 ton/hour to about 40 tons/hour, of about 1 ton/hour to about 50 tons/hour, of about 1 ton/hour to about 75 tons/hour, of about 1 ton/hour to about 100 tons/hour, of about 1 ton/hour to about 125 tons/hour, of about 1 ton/hour to about 150 tons/hour, of about 1 ton/hour to about 175 tons/hour, of about 5 tons/hour to about 200 tons/hour, of about 10 tons/hour to about 200 tons/hour, of about 20 tons/hour to about 200 tons/hour, of about 30 tons/hour to about 200 tons/hour, of about 40 tons/hour to about 200 tons/hour, of about 50 tons/hour to about 200 tons/hour, of about 75 tons/hour to about 200 tons/hour, of about 100 tons/hour to about 200 tons/hour, of about 125 tons/hour to about 200 tons/hour, of about 150 tons/hour to about 200 tons/hour or of about 175 tons/hour to about 200 tons/hour.

In certain embodiments, a reactor for use in the presently disclosed subject matter can be tubular in structure and can have an internal diameter of about 1 meter to about 10 meters. For example, and not by way of limitation, a reactor of the presently disclosed subject matter can have an internal diameter of about 1 meter to about 9 meters, about 1 meter to about 8 meters, about 1 meter to about 7 meters, about 1 meter to about 6 meters, about 1 meter to about 5 meters, about 1 meter to about 4 meters, about 1 meter to about 3 meters, about 1 meter to about 2 meters, about 2 meters to about 10 meters, about 3 meters to about 10 meters, about 4 meters to about 10 meters, about 5 meters to about 10 meters, about 6 meters to about 10 meters, about 7 meters to about 10 meters, about 8 meters to about 10 meters, or about 9 meters to about 10 meters. In certain embodiments, each reactor of the two or more reactors of the presently disclosed subject matter can be of a different scale, size, capacity or structure. For example, but not by way of limitation, reactors with smaller diameters can have improved heat transfer properties and hydrodynamics.

The term “about” or “substantially” means within an acceptable error range for the particular value as determined by one of ordinary skill in the art, which will depend in part on how the value is measure or determine, i.e., the limitations of the measurement system. For example, “about” can mean a range of up to 20%, up to 10%, up to 5% or up to 1% of a given value.

In certain embodiments, the catalyst regenerator 1 of the presently disclosed system 100 can include one or more gas inlet lines 2 that can feed a gas stream, e.g., a regeneration gas, into the regenerator. The gas inlet line can be disposed at any part of the regenerator. For example, but not by way of limitation, the gas inlet 2 can be located at the bottom or the side of the catalyst regenerator to fluidize the catalyst entering the catalyst regenerator from the reactors. In certain embodiments, the regeneration gas is transferred from the gas inlet line 2 to a distributor 9 for distribution and fluidization of the catalyst within the catalyst regenerator. The catalyst regenerator can further include one or more cyclones 13 to separate the effluent gas (e.g., flue gas) from the catalyst in the regenerator 1. The one or more cyclones 13 can be coupled to an exhaust outlet 17 to remove the effluent gas from the catalyst regenerator.

The catalyst regenerator 1 can include one or more transfer lines 4, e.g., regenerated catalyst transfer lines, as described above, to transport regenerated catalyst to the two or more reactors 10. In certain embodiments, the flow of the regenerated catalyst can be controlled by a valve 5 coupled to the transfer line 4. In certain embodiments, the regenerated catalyst can be moved within the transfer line 4 by contact with a lift or carrier gas, as described above, and transported through a nozzle 6 into the reactor.

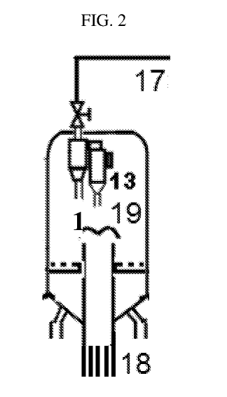

For the purpose of illustration and not limitation, FIG. 2 is a schematic representation of an exemplary catalyst regenerator 1, in accordance with one non-limiting embodiment of the disclosed subject matter. In certain embodiments, the catalyst regenerator 1 can include one or more cyclones 13 to separate the effluent gas from the catalyst in the catalyst regenerator 1. The one or more cyclones 13 can be coupled to gas exhaust line 17 to remove the effluent gas from the catalyst regenerator 1. In certain embodiments, the transfer lines coupled to the two or more reactors and the catalyst regenerator 1 to transfer spent catalyst can merge to form a single transfer line 18 at the base of the catalyst regenerator 1. The merged single transfer line 18 can be coupled to a distributor 19 to distribute the catalyst in the catalyst regenerator 1. In certain embodiments, the distributor 19 is an open-hat distributor.

The catalyst regenerator 1 can also include additional components and accessories including, but not limited to, reaction zones, heating elements, pH meters, pressure indicators, pressure transmitters, thermowells, temperature-indicating controllers, gas detectors, analyzers and viscometers. The components and accessories can be placed at various locations on the catalyst regenerator 1.

The two or more reactors 10 of a system 100 of the presently disclosed subject matter can further include additional components and accessories including, but not limited to, one or more gas exhaust lines, fresh catalyst inlet lines, reaction zones and heating elements. The reactors of the system 100 of the present disclosure can also include one or more measurement accessories. The one or more measurement accessories can be any measurement accessory known to one of ordinary skill in the art including, but not limited to, pH meters, pressure indicators, pressure transmitters, thermowells, temperature-indicating controllers, gas detectors, analyzers and viscometers. The components and accessories can be placed at various locations on the reactors 1 0.

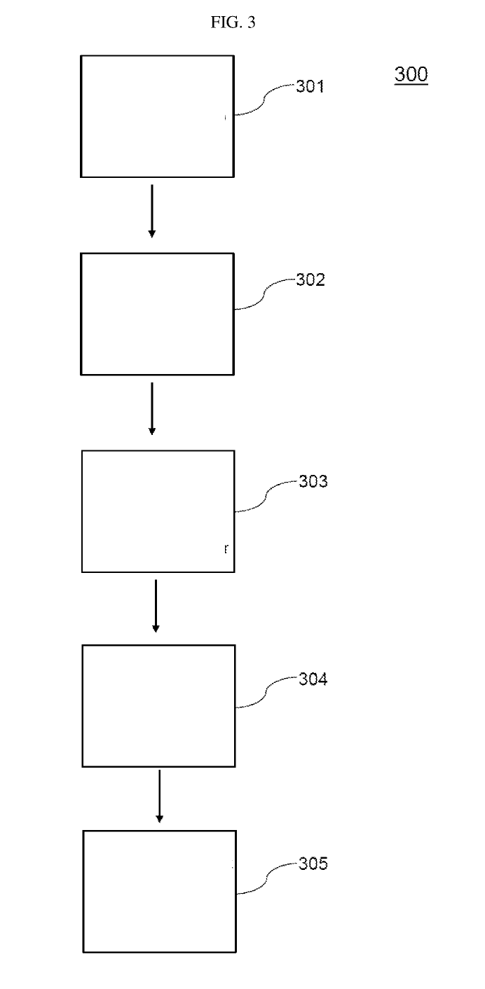

The presently disclosed subject matter further provides for methods of dehydrogenating alkanes to generate unsaturated hydrocarbons such as olefins. For the purpose of illustration and not limitation, FIG. 3 shows an exemplary method 300 for the dehydrogenation of alkanes in accordance with one embodiment of the disclosed subject matter. The method of dehydrogenating alkanes 300 can include providing a hydrocarbon feedstream into the two or more reactors 301. For example, but not by way of limitation, the hydrocarbon feedstream can be introduced into two or more fluidized bed reactors through feed lines.

In certain embodiments, the hydrocarbon feedstream includes one or more alkanes. Non-limiting examples of alkanes include linear alkanes, branched alkanes, lower alkanes (i.e., alkanes having eight or fewer carbon atoms) and higher alkanes (i.e., alkanes have nine or more carbon atoms). In certain embodiments, the one or more alkanes can include ethane, propane, n-butane, iso-butane, pentane, iso-pentane, neopentane, hexane, 2,2-dimethylbutane (neo-hexane), 2-methylpentane (iso-hexane), C₂-C₇ linear hydrocarbons, C₂C₇ branched hydrocarbons, C₈-C₁₉ linear hydrocarbons, C₈-C₁₉ branched hydrocarbons or a combination comprising at least one of the foregoing. Additional non-limiting examples of alkanes include other compounds that contain saturated hydrocarbon moieties (e.g., a —CH₂—CH₂-moiety) capable of dehydrogenation to an alkene moiety. In certain embodiments, the hydrocarbon mixture can include alkanes that incorporate moieties other than saturated hydrocarbons, e.g., unsaturated hydrocarbon moieties and/or heteroatoms.

In certain embodiments, the hydrocarbon feedstream can include additional components. For example, but not by way of limitation, the hydrocarbon feedstream can further include other gases that do not negatively affect the reaction, e.g., inert gases. Non-limiting examples of other gases include steam, water, nitrogen gas (N2), helium (He), carbon monoxide (CO), carbon dioxide (CO₂) and ethane. In certain embodiments, the CO and/or CO₂ gas can act as a fluffing gas. In certain embodiments, the hydrocarbon mixture feedstream includes iso-butane. In certain embodiments, the hydrocarbon mixture feedstream includes propane.

The method can further include reacting the hydrocarbon feedstream with a catalyst to produce an olefin product through a catalytic dehydrogenation reaction in the two or more reactors 302. Non-limiting examples of the olefins that can be produced by the dehydrogenation reaction include ethylene, iso-butylene, propylene, C₂ to C₄ monoolefins, C₂ to C₆ monoolefins, C₆ to C₁₉ monoolefins or a combination comprising at least one of the foregoing. In certain embodiments, each of the two or more reactors can be configured to produce the same olefin, or alternatively, each reactor can produce different olefins that correspond to the alkanes present in the hydrocarbon mixture. For example, but not by way of limitation, one of the two or more reactors can be configured to perform a dehydrogenation reaction of iso-butane to iso-butylene, and a second reactor of the two or more reactors can be configured to perform a dehydrogenation reaction of propane to propylene.

Dehydrogenation reactions of alkanes can be thermodynamically favored at high temperatures and low pressures. In certain embodiments, the dehydrogenation reaction can occur at an operating temperature of about 400° C. to about 700° C. In certain embodiments, the dehydrogenation reaction can occur at a pressure of about 5 atmospheres (atms) or lower (about 506 kiloPascals (kPa) or lower). In certain embodiments, the dehydrogenation reaction can occur at a pressure of about 0.01 atms to about 5 atms (about 1.01 kPa to about 506 kPa).

In certain embodiments, the olefin being produced by a dehydrogenation reaction can be iso-butylene and/or propylene. In certain embodiments, the dehydrogenation reaction can be the dehydrogenation of iso-butane to iso-butylene. In certain embodiments, the dehydrogenation reaction can be the dehydrogenation of cyclohexane to benzene. In certain embodiments, the dehydrogenation reaction can be the dehydrogenation of propane to propylene.

The method can further include removing and transferring the spent (e.g., coked) catalyst from the two or more reactors to a catalyst regenerator 303. In certain embodiments, the catalyst is transferred to the catalyst regenerator from each of the reactors by a transfer line. In certain embodiments, the regenerated catalyst can be moved within the transfer line by contact with a carrier or lift gas. The carrier or lift gas, as previously described herein, can include, but is not limited to, natural gas, air, oxygen-rich gas, oxygen-lean gas, carbon monoxide, carbon dioxide, nitrogen, steam combustion or exhaust gas, or a combination comprising at least one of the foregoing. In certain embodiments, the carrier gas includes air.

The method can further include regenerating the catalyst in the catalyst regenerator to obtain a regenerated catalyst by exposing the spent catalyst to a regeneration gas stream to remove coke, e.g., hydrocarbon, from the surface of the deactivated catalyst 304. For example, but not by way of limitation, the hydrocarbon deposits on the deactivated catalyst can be oxidized in the presence of regeneration gas to form a regenerated catalyst and a regenerator effluent gas (e.g., flue gas). In certain embodiments, the flue gas can include carbon dioxide, carbon monoxide, hydrogen, nitrogen or a combination comprising at least one of the foregoing. In certain embodiments, the regeneration gas stream can include oxygen, air, steam, hydrocarbons, fuel gas or a combination comprising at least one of the foregoing. In certain embodiments, the regeneration mixture can be steam alone, in the absence of oxygen. In certain embodiments, oxygen can be supplied in the form of air. Alternatively or additionally, the oxygen source can be a more concentrated oxygen source. In certain embodiments, the composition of the regeneration mixture can vary during a regeneration process.

In certain embodiments, the spent catalyst can be subjected to contact with the regeneration gas at temperatures of about 400° C. to about 700° C. to remove the residual hydrocarbon and coke deposits from the catalyst. In certain embodiments, the contact temperature can be about 400° C. to about 425° C., about 400° C. to about 450° C., about 400° C. to about 475° C., about 400° C. to about 500° C., about 400° C. to about 525° C., about 400° C. to about 550° C., about 400° C. to about 575° C., about 400° C. to about 600° C., about 400° C. to about 625° C., about 400° C. to about 650° C., about 400° C. to about 675° C., about 425° C. to about 700° C., about 450° C. to about 700° C., about 475° C. to about 700° C., about 500° C. to about 700° C., about 525° C. to about 700° C., about 550° C. to about 700° C., about 575° C. to about 700° C., about 600° C. to about 700° C., about 625° C. to about 700° C., about 650° C. to about 700° C. or about 675° C. to about 700° C.

The duration of the regeneration process can vary widely and is dependent on the degree of decoking that is desired. For example, the time during which the spent catalyst is exposed to the regeneration gas can be about 1 minute to about 100 minutes. In certain embodiments, the contact time can be about 1 minute to about 10 minutes, about 1 minute to about 20 minutes, about 1 minute to about 30 minutes, about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 10 minutes to about 100 minutes, about 20 minutes to about 100 minutes, about 30 minutes to about 100 minutes, about 40 minutes to about 100 minutes, about 50 minutes to about 100 minutes, about 60 minutes to about 100 minutes, about 70 minutes to about 100 minutes, about 80 minutes to about 100 minutes, or about 90 minutes to about 100 minutes.

In certain embodiments, the regeneration of spent catalyst can result in the oxidation of the metals of the catalyst. As a result, the regeneration of the catalyst can further include contacting the oxidized catalyst with a reducing gas stream to reduce the catalyst. For example and not by way of limitation, the reducing gas stream can include hydrogen, nitrogen or a combination comprising at least one of the foregoing. In certain embodiments, the regenerated catalyst can undergo reduction from about 1 second to about 60 minutes.

The regenerated catalyst can then be transferred from the catalyst regenerator to the two or more reactors (e.g., fluidized bed reactors) to be used in the dehydrogenation of alkanes 305. In certain embodiments, the regenerated catalyst can be transferred to each of the two or more reactors through a transfer line. In certain embodiments the regenerated catalyst can be separated from the regeneration gas and effluent gas (e.g., flue gas) by one or more cyclone separators prior to the transport of the catalyst to the reactors. In certain embodiments, the oxidized catalyst can undergo reduction while being transported to the reactor within the transport line. For example, if the regenerated catalyst is moved within the transfer line by contact with a carrier or lift gas, the carrier or lift gas can also function to reduce the oxidized catalyst, e.g., by the use of hydrogen or nitrogen-based gases.

In certain embodiments, the spent catalyst can be stripped in a reactor stripper prior to transfer of the spent catalyst to the catalyst regenerator. To maintain catalyst activity, the reactor stripper functions to at least partially remove the hydrocarbon material that is associated with the deactivated catalyst prior to entry into the catalyst regenerator. In certain embodiments, “at least partially remove” can include the removal of about 5%, about 10%, about 20%, about 30%, about 40%, about 50%, about 60%, about 70% or more of the coke from the surface of the catalyst during the stripping process. In certain embodiments, hydrocarbon on the surface and within the pores of the deactivated catalyst can be removed within the reactor stripper by contact with a stripping gas. Non-limiting examples of a stripping gas include nitrogen, carbon dioxide, water vapor, recycle gas, obtained as exhaust from the chemical reaction, or a combination comprising at least one of the foregoing. Catalyst residence time in the stripper can be about 1 second to about 200 seconds. In certain embodiments, the total catalyst residence time in the reactors and/or regenerator can be about 1 minute to about 100 minutes.

The catalysts to be used in the method of the disclosed subject matter can be any catalyst known to one of ordinary skill in the art that can be used to catalyze the dehydrogenation of alkanes In certain embodiments, catalyst compositions for catalyzing dehydrogenation of alkanes include oxides, carbides, hydroxides of suitable metals, or a combination comprising at least one of the foregoing. Non- limiting examples of suitable metals include chromium (Cr), copper (Cu), manganese (Mn), potassium (K), palladium (Pd), cobalt (Co), cerium (Ce), tungsten (W), platinum (Pt), sodium (Na), nickel (Ni), osmium (Os), ruthenium (Ru), rhodium (Rh), iridium (Ir), tin (Sn), cesium (Cs), or a combination comprising at least one of the foregoing.

In certain embodiments, the catalysts for use in alkane dehydrogenation reactions can include a solid support. Suitable supports can be any support materials, which exhibit good stability at the reaction conditions of the disclosed methods, and are known by one of ordinary skill in the art. Non-limiting examples of solid supports include various metal salts, metalloid oxides, and metal oxides, e.g., titania (titanium oxide), zirconia (zirconium oxide), silica (silicon oxide), alumasina (aluminum oxide), zeolites, magnesia (magnesium oxide), magnesium chloride, or a combination comprising at least one of the foregoing.

U.S. Pat. Nos. 6,486,220, 8,551,434 and 8,288,446, incorporated herein by reference in their entireties, disclose catalysts that can be used in the disclosed subject matter. Additional non-limiting examples of catalyst compositions include Pt—Sn/Al₂O₃, Pt—Sn/SAPO-34, Pt—Sn/ZSM-5, Pt—Sn/Zeolites, Cr₂O₃, Cr₂O₃/Al₂O₃, Cr/ZrO₂, Cr/Al₂O₃, Cr/SiO₂, Cu-Mn/Al₂O₃, Cr/MgO, as well as combinations comprising at least one of the foregoing. A non-limiting example of a commercially available Cr/Al₂O₃ catalyst is CATOFIN® (Süd-Chemie AG, Munich, Germany)

In certain embodiments, the catalyst compositions of the present disclosure further include one or more promoters. Promoters function to increase the activity and/or selectively of the catalyst metal. Non-limiting examples of suitable promoters includes lanthanides, alkaline earth metals, rare earth metals, magnesium, tin, rhenium and alkali metals such as lithium, sodium, potassium, rubidium, cesium, or a combination comprising at least one of the foregoing. In certain embodiments, the catalyst can include tin as a promoter. Additionally, the catalyst can contain at least one co- promoter component such as rhenium, sulphur, molybdenum, tungsten, chromium, or a combination comprising at least one of the foregoing.

The catalyst used in the present disclosure can be of any shape and size. For example, but not by way of limitation, the catalyst can be in the form of powder, granules, spheres, pellets, beads, cylinders, trilobe and quadralobe shaped pieces. In certain embodiments, the catalyst is in the form of a powder.

The catalyst used in the present disclosure can be prepared by any catalyst synthesis process well known in the art. See, for example, U.S. Pat. Nos. 6,299,995, 6,293,979 and 8,288,446, each of which is incorporated herein by reference in its entirety. Additional examples include, but are not limited to, spray drying, precipitation, impregnation, incipient wetness, ion exchange, fluid bed coating, physical or chemical vapor deposition.

The processes and systems of the presently disclosed subject matter can have numerous advantages over existing technology, including improved heat and mass transfer, improved control of temperature and product profile, reduced downtime and simplified operation.

The systems and methods disclosed herein include at least the following embodiments:

Embodiment 1: A system for the catalytic dehydrogenation of alkanes, comprising: two or more reactors configured to perform a dehydrogenation reaction of an alkane in the presence of a catalyst to produce an olefin; and a catalyst regenerator, coupled to each of the two or more reactors through at least one spent catalyst transfer line, for regeneration of spent catalyst transferred from the two or more reactors.

Embodiment 2: The system of Embodiment 1, wherein the two or more reactors comprise fluidized bed reactors.

Embodiment 3: The system of Embodiment 1 or Embodiment 2, wherein each of the two or more reactors include a single catalyst bed.

Embodiment 4: The system of any of the preceding embodiments, further comprising two or more regenerated catalyst transfer lines coupled to the catalyst regenerator and the two or more reactors for the transfer of the regenerated catalyst from the catalyst regenerator to the two or more reactors.

Embodiment 5: The system of any of the preceding embodiments, wherein the catalyst comprises platinum, chromium, or a combination comprising at least one of the foregoing.

Embodiment 6: The system of any of the preceding embodiments, wherein the two or more reactors are configured to perform different alkane dehydrogenation reactions.

Embodiment 7: The system of any of the preceding embodiments, wherein the alkane comprises a C₂ to a C₇ alkane.

Embodiment 8: The system of any of the preceding embodiments, wherein the alkane is ethane, butane, iso-butane, propane, iso-propane, neo-propane, hexane, heptane, or a combination comprising at least one of the foregoing.

Embodiment 9: The system of any of the preceding embodiments, wherein the alkane comprises isobutane and the olefin comprises iso-butylene.

Embodiment 10: The system of any of the preceding embodiments, wherein the alkane comprises propane and the olefin comprises propylene.

Embodiment 11: A method for producing an olefin, comprising: feeding a hydrocarbon feedstream comprising an alkane into two or more reactors; reacting the hydrocarbon feedstream with a catalyst to produce an olefin in each of the two or more reactors through a dehydrogenation reaction; removing and transferring spent catalyst from the two or more reactors to a catalyst regenerator; regenerating the spent catalyst in the catalyst regenerator to obtain a regenerated catalyst; and transferring the regenerated catalyst to the two or more reactors to be used in the dehydrogenation reaction.

Embodiment 12: The method of Embodiment 11, further comprising stripping coke from the surface of the spent catalyst prior to the transfer of the spent catalyst to the catalyst regenerator from the two or more reactors.

Embodiment 13: The method of Embodiment 11 or Embodiments 12, wherein the alkane comprises a C₂ to a C₇ alkane

Embodiment 14: The method of any of the preceding embodiments, wherein the alkane is ethane, butane, iso-butane, propane, iso-propane, neo-propane, hexane, heptane, or a combination comprising at least one of the foregoing.

Embodiment 15: The method of any of the preceding embodiments, wherein the catalyst comprises platinum, chromium, or a combination comprising at least one of the foregoing.

Embodiment 16: The method of any of the preceding embodiments, wherein the alkane comprises iso-butane and the olefin comprises iso-butylene.

Embodiment 17: The method of any of the preceding embodiments, wherein the alkane comprises propane and the olefin comprises propylene.

Embodiment 18: The method of any of the preceding embodiments, wherein each of the two or more reactors produce different olefins.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A system for the catalytic dehydrogenation of alkanes, comprising:
two or more reactors configured to perform a dehydrogenation reaction of an alkane in the presence of a catalyst to produce an olefin; and
a catalyst regenerator, coupled to each of the two or more reactors through at least one spent catalyst transfer line, for regeneration of spent catalyst transferred from the two or more reactors.

2. The system of claim 1, wherein the two or more reactors comprise fluidized bed reactors.

3. The system of claim 1, wherein each of the two or more reactors include a single catalyst bed.

4. The system of claim 1, further comprising two or more regenerated catalyst transfer lines coupled to the catalyst regenerator and the two or more reactors for the transfer of the regenerated catalyst from the catalyst regenerator to the two or more reactors.

5. The system of claim 1, wherein the catalyst comprises platinum, chromium, or a combination comprising at least one of the foregoing.

6. The system of claim 1, wherein the two or more reactors are configured to perform different alkane dehydrogenation reactions.

7. The system of claim 1, wherein the alkane comprises a C₂ to a C₇ alkane.

8. The system of claim 1, wherein the alkane is ethane, butane, iso-butane, propane, iso-propane, neo-propane, hexane, heptane, or a combination comprising at least one of the foregoing.

9. The system of claim 1, wherein the alkane comprises isobutane and the olefin comprises iso-butylene.

10. The system of claim 1, wherein the alkane comprises propane and the olefin comprises propylene.

11. A method for producing an olefin, comprising:
feeding a hydrocarbon feedstream comprising an alkane into two or more reactors;
reacting the hydrocarbon feedstream with a catalyst to produce an olefin in each of the two or more reactors through a dehydrogenation reaction;
removing and transferring spent catalyst from the two or more reactors to a catalyst regenerator;
regenerating the spent catalyst in the catalyst regenerator to obtain a regenerated catalyst; and
transferring the regenerated catalyst to the two or more reactors to be used in the dehydrogenation reaction.

12. The method of claim 11, further comprising stripping coke from the surface of the spent catalyst prior to the transfer of the spent catalyst to the catalyst regenerator from the two or more reactors.

13. The method of claim 11, wherein the alkane comprises a C₂ to a C₇ alkane.

14. The method of claim 11, wherein the alkane is ethane, butane, iso-butane, propane, iso-propane, neo-propane, hexane, heptane, or a combination comprising at least one of the foregoing.

15. The method of claim 11, wherein the catalyst comprises platinum, chromium, or a combination comprising at least one of the foregoing.

16. The method of claim 11, wherein the alkane comprises iso-butane and the olefin comprises iso-butylene.

17. The method of claim 11, wherein the alkane comprises propane and the olefin comprises propylene.

18. The method of claim 11, wherein each of the two or more reactors produce different olefins.
